(19) United States
(12) Plant Patent Application Publication
(10) Pub. No.: US 2005/0216983 P1
(43) Pub. Date: Sep. 29, 2005

(54) Mango tree named 'B74'

(50) MANGIFERA INDICA; MANGO

(76) Inventors: Anthony William Whiley, Nambour (AU); John W. Dorrian, Childers (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/811,483

(22) Filed: Mar. 26, 2004

(51) Int. Cl. A01H 5/00

(52) U.S. Cl. PLT/159

(57) ABSTRACT

A new and distinct mango tree variety that possesses late season maturing fruits with predominately yellow and red skin, and pale yellow, firm flesh having a low amount of non-fleshy fiber attached to the stone.

### LATIN NAME OF THE GENUS AND SPECIES OF THE PLANT CLAIMED

Mangifera indica

### VARIETY DENOMINATION

‘B74’

### BACKGROUND AND SUMMARY OF THE INVENTION

This invention relates to the discovery and asexual propagation of a new variety of mango tree, as herein described and illustrated. The new variety was first hybridized by controlled pollination. The new variety is a precocious, heavy-cropping, upright tree yielding red-skinned, medium-sized, terpinolene-flavored fruit.

The seed parent is ‘Sensation’ and the pollen parent is ‘Kensington Pride.’ The new variety was selected and evaluated at the fruiting stage on the property of Mr. And Mrs. L. W. Dorrian at Childers, in Queensland, Australia.

The new mango tree variety was first asexually propagated by grafting onto seedling rootstocks in Childers, in Queensland, Australia.

The new mango tree variety cv. ‘B74’ has been shown to maintain its distinguishing characteristics through successive asexual propagations.

### BRIEF DESCRIPTION OF THE PHOTOGRAPHS

The accompanying photographic illustrations illustrate in full color the new mango variety ‘B74.’ The colors are as nearly true as is reasonably possible in a color representation of this type.

FIG. 1 is a photograph of the fruit of the new mango variety ‘B74’ (center) and comparators ‘Kensington Pride’ (left) and ‘R2E2’ (right) showing differences in size and color.

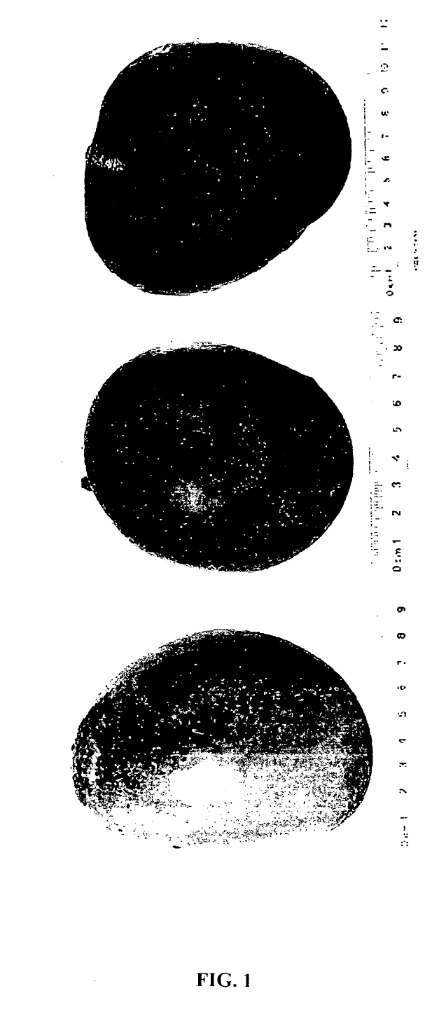

FIG. 2 is a photograph of the tree of the new mango variety ‘B74.’

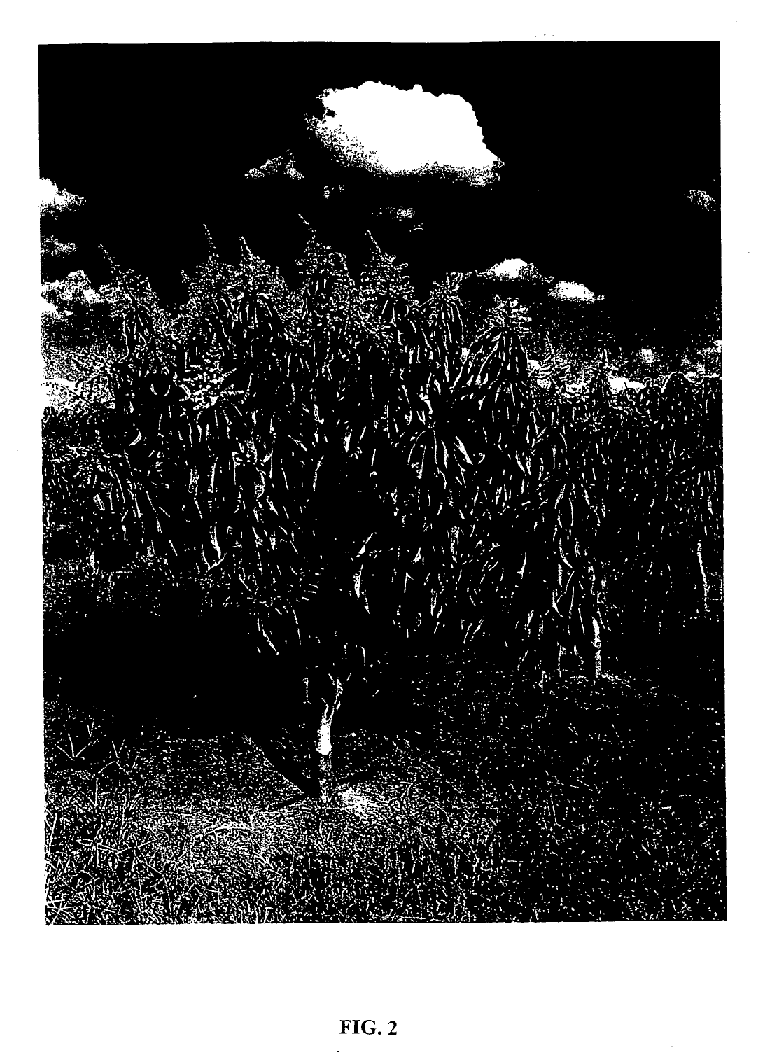

FIG. 3 is a photograph of the floral panicle of the new mango variety ‘B74.’

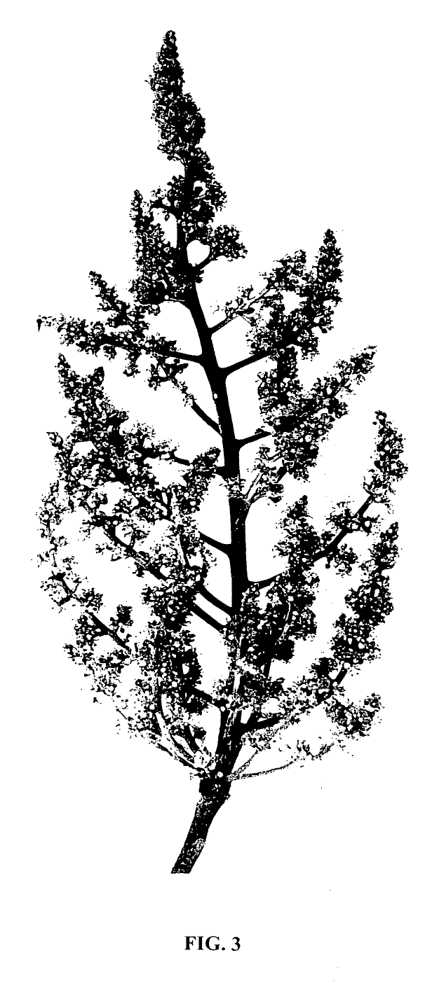

FIG. 4 is a photograph of flowers and floral buds of the new mango variety ‘B74.’

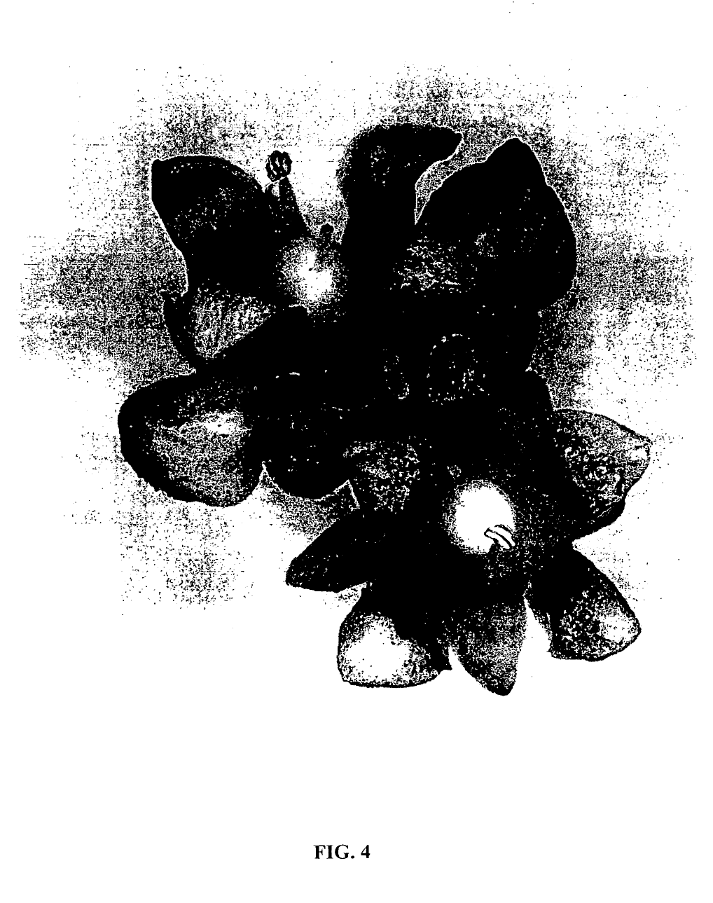

FIG. 5 is a photograph of a single flower of the new mango variety ‘B74.’

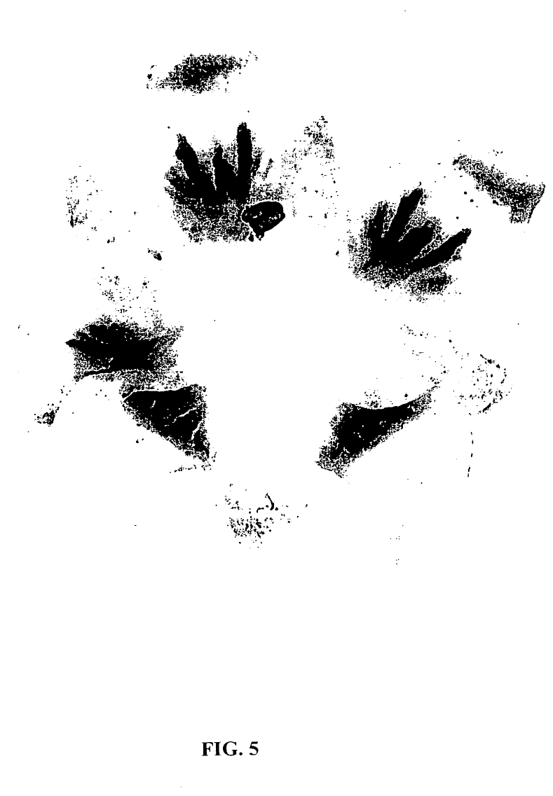

FIG. 6 is a photograph of a cross-sectional slice of the fruit of the new variety ‘B74,’ sliced through the fruit in the region above the seed.

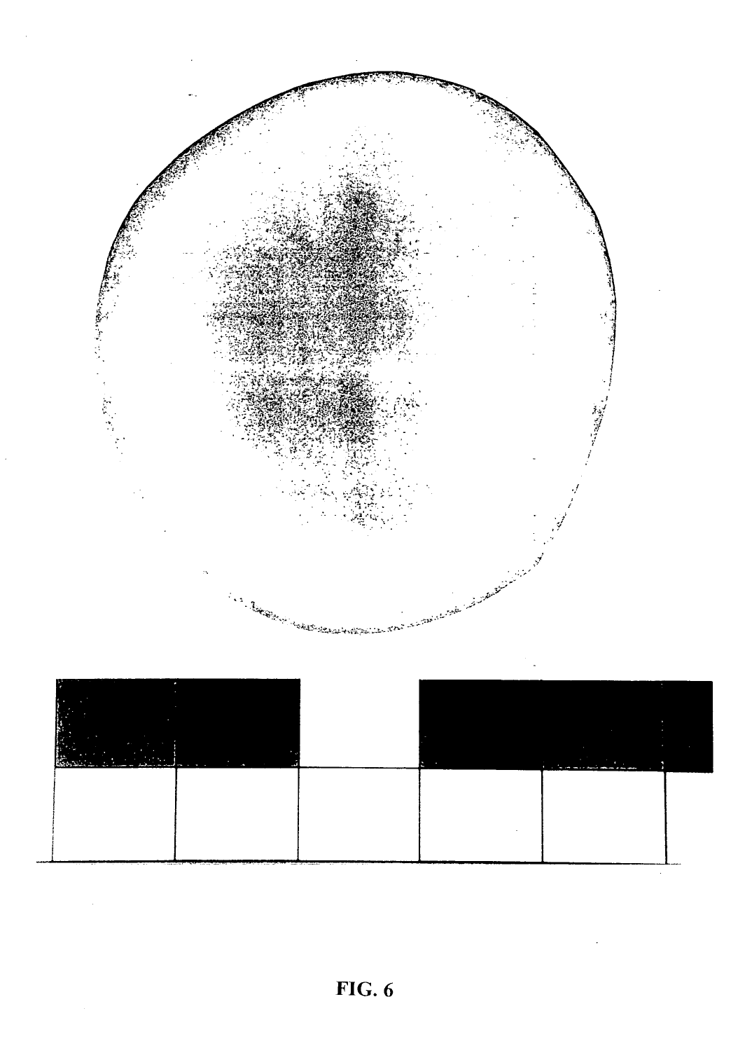

FIG. 7 is a photograph of the seed of the new mango variety ‘B74.’

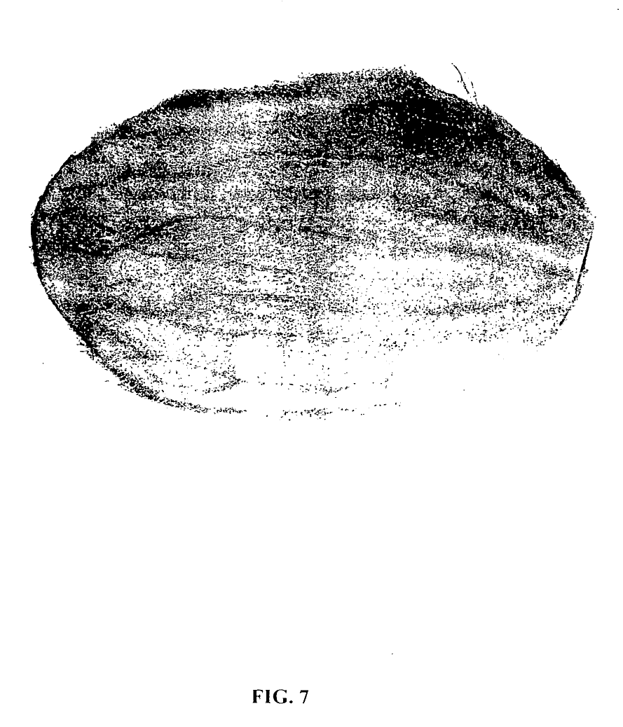

### DETAILED BOTANICAL DESCRIPTION OF THE INVENTION

Throughout this specification, color names beginning with a small letter signify that the name of that color, as used in common speech, is aptly descriptive.

The descriptive matter which follows pertains to ‘B74’ mango trees (as well as the comparative varieties ‘Sensation,’ ‘Kensington Pride,’ and ‘R2E2’) grown in the vicinity of Childers, Queensland, Australia. The scions of the candidate and comparator varieties were topworked to ‘Keitt’ trees that were originally grafted to polyembryonic seedlings of ‘Kensington Pride.’ Ten single tree replicates of each cultivar were planted at 6×10 m intervals in red basaltic soil (kraznozem) following a completely randomised design. Pest and disease treatments were applied as required. Irrigation and fertilizer application followed commercial practice. 10-20 random measurements of each characteristic were obtained from each replicate. Redness of skin color was determined using a Minolta Chroma Meter CR-200 to measure the hue angle (H). Mean values were taken from measurements at three points from the shoulder to the basal end of the sun-exposed side of each fruit. The lower the hue angle, the greater the red coloration. The observations described herein are believed to apply to plants of the variety grown under similar conditions of soil and climate elsewhere.

The new mango tree cultivar, ‘B74’ is quite distinct from its seed parent ‘Sensation’, and may be distinguished from its seed parent in the following characteristics: The average fruit weight of ‘B74’ (457 g) is larger than ‘Sensation’ (360 g). Additionally, the new mango tree cultivar matures 3-4 weeks earlier than the seed parent ‘Sensation’, which is a very late variety. The skin color of the new mango tree cultivar is red and yellow, while the skin color of the seed parent ‘Sensation’ has a bright yellow background with a dark red to purple blush that covers most of the surface. Further, while the new mango tree cultivar has a terpinolene aroma in both the leaves and fruit, the seed parent ‘Sensation’ has no distinguishable terpinolene aroma in either leaves or fruit.

The new mango tree cultivar may be distinguished from the pollen parent, ‘Kensington Pride,’ by the following characteristics. The tree of the new mango cultivar ‘B74’ has a more erect form and lower vigor than the tree of the pollen parent ‘Kensington Pride.’ The fruit of the new mango tree cultivar matures in late season, while the fruit of ‘Kensington Pride’ matures early to mid-season. The predominant skin color of ripe fruit of the new mango tree cultivar is red and yellow, while the predominant skin color of ripe fruit of ‘Kensington Pride’ is yellow and red. The fruit flesh of the new mango tree cultivar is pale yellow in color, while the fruit flesh of ‘Kensington Pride’ is yellow. The fruit shape of the new mango tree cultivar is broad elliptic, while fruit shape of the pollen parent ‘Kensington Pride’ is medium elliptic. The sinus proximal of the stylar scar of the new mango tree cultivar is absent, while it is present in the pollen parent ‘Kensington Pride.’ The fruit of the new mango tree cultivar is somewhat smaller than that of ‘Kensington Pride’ (457 g as compared to 475 g). The seed of the new mango tree cultivar is of the monoembryonic type, while the seed of the pollen parent ‘Kensington Pride’ is polyembryonic.

The new mango tree variety ‘B74’ may be distinguished from presently available cultivars. The new mango tree cultivar ‘B74’ can be compared, for example, to ‘R2E2’, which is commonly grown in Australia, by the following distinguishing characteristics: The leaves of the new mango tree variety are shorter and wider than the leaves of ‘R2E2.’ The new mango tree cultivar has a higher percentage of bunch bearing inflorescences than ‘R2E2.’ The fruit of ‘B74’ is smaller than that of ‘R2E2.’ The flesh color of the new mango tree variety is pale yellow, while the flesh color of ‘R2E2’ is yellow. The skin color of ripe fruit of the new mango tree variety is predominantly red and yellow, while the predominant skin color of ripe fruit of ‘R2E2’ is predominantly yellow and red. Additional comparative information can be found in Table 1.

### Tree

General:
   Vigor.—low to moderate.
   Density of foliage.—open.
   Shape.—upright.
   Form.—erect.
   Fruit bearing.—mid-late season maturity.
   Root stock.—‘Kensington Pride’.

### Leaves

Young leaf:
   Anthocyanin.—present.
   Anthocyanin hue.—red.
   Intensity of color.—strong.
Fully expanded leaf:
   Average length.—approximately 204 mm.
   Average width.—approximately 62 mm.
   length/width ratio.—low (approximately 3.5).
   Attitude.—horizontal.
   Leafsurface.—smooth.
   Profile.—concave cross section.
   Shape.—elliptic.
   Cross-section shape.—concave.
   Leaf blade tip.—acuminate.
   Base.—acute.
   Symmetry.—asymmetric.
   Undulation of margin.—not undulated.
   Curvature of midrib.—present.
   Position of midrib curvature.—apical.
   Twisting of blade.—absent.
   Aroma present when crushed.—terpinolene aroma.
   Relief of upper surface of leaf.—slightly sunken between veins.
   Length of petiole.—approximately 40 mm.

### Inflorescence

Percentage of bunch bearing inflorescences.—approximately 66%.

### Fruit

General harvest characteristics:
   General fruiting characteristics.—bunch bearing with 2-4 fruits commonly carried on each inflorescence.
   Season maturity.—mid-late season maturity.

### Mature Fruit

General:
   Length.—medium — approximately 101.3 mm.
   Width.—medium — approximately 91.3 mm.
   Length/width ratio.—medium — approximately 1.1.
   Shape.—ovate.
   Cross-sectional shape.—broad elliptic.
   Average weight.—approximately 457 g.
   Stalk cavity.—shallow.
   Sinus proximal of stylar scar.—absent.
   Bulge proximal of stylar scar.—absent.
Skin of mature fruit:
   Anthocyanin coloration.—skin develops high levels of red anthocyanin pigmentation where exposed to sun.

### Ripe Fruit

Skin of ripe fruit:
   Redness of skin color (hue angle).—approximately 44.7.
Flesh of ripe fruit:
   Main color of flesh.—pale yellow.
   Firmness of flesh.—firm.
   Texture of flesh.—smooth.
   Amount of non-fleshy fiber in flesh attached to stone.—low.
   Turpinolene flavor.—present.
   Sap exudation at harvest.—present with sap bum and skin browning.

### Seed

Size.—small.

Polyembryony.—monoembryonic.

TABLE 1
COMPARISON OF ‘B74’ WITH OTHER MANGO VARIETIES

| | | ‘Kensington | |
|---|---|---|---|
| | ‘B74’ | Pride’ | ‘R2E2’ |
| YOUNG LEAF | | | |
| Anthocyanin | present | present | present |
| Anthocyanin hue | red | red | red |
| Intensity of color | strong | strong | strong |
| MATURE LEAF | | | |
| Terpinolene aroma | present | present | absent |
| Cross-section shape | concave | concave | straight |
| Relief of upper | slightly sunken | raised between | raised between |
| surface | between veins | veins | veins |
| Shape of tip | acuminate | attenuate | acuminate |
| Shape of base | acute | acute | rounded |
| Predominant shape | elliptic | elliptic | elliptic |
| Symmetry | asymmetric | asymmetric | asymmetric |
| Curvature of midrib | present | present | present |
| Attitude | horizontal | horizontal | horizontal |
| Petiole length (mm) | | | |
| Mean | 39.8 | 21.3 | 35.5 |
| std deviation | 0.7 | 0.7 | 1.0 |
| LSD/sig | 0.6 | P ≦ 0.01 | P ≦ 0.01 |
| Lamina length (mm) | | | |
| Mean | 203.9 | 181.5 | 236.8 |
| std deviation | 3.9 | 6.2 | 4.4 |
| LSD/sig | 2.0 | P ≦ 0.01 | P ≦ 0.01 |
| Lamina width (mm) | | | |
| Mean | 62.3 | 39.7 | 51.8 |
| std deviation | 0.8 | 1.1 | 0.8 |
| LSD/sig | 1.30 | P ≦ 0.01 | P ≦ 0.01 |
| Length/width ratio | | | |
| Mean | 3.44 | 4.00 | 4.00 |
| std deviation | 0.89 | 1.11 | 0.96 |
| LSD/sig | 0.21 | P ≦ 0.01 | P ≦ 0.01 |
| INFLORESCENCE | | | |
| Percentage of | | | |
| bunch-bearing | | | |
| inflorescences | | | |
| Mean | 66.4 | 22.9 | 34.3 |
| std deviation | 9.0 | 9.1 | 10.1 |
| LSD/sig | 11.7 | P ≦ 0.01 | P ≦ 0.01 |
| MATURE FRUIT | | | |
| Cross-sectional | broad elliptic | medium elliptic | broad elliptic |
| shape | | | |
| Depth of stalk | shallow | medium | medium |
| cavity | | | |
| Sinus proximal of | absent | present | absent |
| stylar scar | | | |
| Bulge proximal of | absent | absent | absent |
| stylar scar | | | |
| Ripe fruit: | red and yellow | yellow and red | yellow and red |
| predominant | | | |
| skin color | | | |
| Ripe fruit: | pale yellow | yellow | yellow |
| predominant | | | |
| flesh color | | | |
| Ripe fruit: | low | medium | low |
| amount of | | | |
| fiber in flesh | | | |
| attached to | | | |
| stone | | | |
| Ripe fruit: | present | present | absent |
| terpinolene | | | |
| flavor | | | |
| Length (mm) | | | |
| Mean | 101.23 | 113.52 | 117.11 |
| std deviation | 2.68 | 2.14 | 3.34 |
| LSD/sig | 3.43 | P ≦ 0.01 | P ≦ 0.01 |
| Width (mm) | | | |
| Mean | 91.28 | 87.94 | 111.98 |
| std deviation | 2.80 | 2.71 | 2.43 |
| LSD/sig | 3.17 | P ≦ 0.01 | P ≦ 0.01 |
| Length/Width ratio | | | |
| mean | 1.11 | 1.29 | 1.05 |
| std deviation | 0.01 | 0.03 | 0.01 |
| LSD/sig | 0.02 | P ≦ 0.01 | P ≦ 0.01 |
| Weight (g) | | | |
| mean | 457.4 | 475.1 | 802.7 |
| std deviation | 38.1 | 37.0 | 53.0 |
| LSD/sig | 50.5 | ns | P ≦ 0.01 |
| *Ripe color | | | |
| (hue angle) | | | |
| mean | 44.73 | 67.36 | 53.50 |
| std deviation | 4.18 | 2.05 | 2.58 |
| LSD/sig | 3.41 | P ≦ 0.01 | P ≦ 0.01 |
| Embryonic type | monoembryonic | Polyembryonic | mostly |
| | | | polyembryonic |
| TREE | | | |
| Form | erect | Spreading | erect |
| Vigor | low-moderate | high | moderate |
| Fruit maturity | late | Early-mid | mid-late |
| season | | | |

*Redness of skin color was determined using a Minolta Chroma Meter CR-200 to measure the hue angle (H).

1. A new and distinct mango tree named ‘B74’ as herein described and illustrated.
